(19) United States
(12) Patent Application Publication
(10) Pub. No.: US 2025/0127415 A1
(43) Pub. Date: Apr. 24, 2025

(54) SYSTEM AND METHOD FOR MINIMIZING COAXIAL CABLE EFFECTS ON MEASUREMENTS IN ELECTRICAL IMPEDANCE TOMOGRAPHY

(71) Applicant: The Research Foundation for the State University of New York, Albany, NY (US)

(72) Inventors: Gary Saulnier, Albany, NY (US); Omid Rajabi Shishvan, Albany, NY (US); Ahmed Abdelwahab, Morrisville, NC (US)

(21) Appl. No.: 18/920,713

(22) Filed: Oct. 18, 2024

Related U.S. Application Data
(60) Provisional application No. 63/544,670, filed on Oct. 18, 2023.

(51) Int. Cl. A61B 5/0536

(52) U.S. Cl. CPC A61B 5/0536

(57) ABSTRACT

A system and method for minimizing signal interference in coaxial cables carrying signals for electrical impedance tomography (EIT) by compensating for losses from both the series and shunt elements on the path from the source to the load, providing a compensated load current and voltage. One or a series of two-port networks is used to modify a current source with applied current to compensate for losses in the cable to attain the desired load current. The input current is adjusted using the measured voltage and the shunting output and stray impedance, preferably determined during a calibration process.

### CROSS-REFERENCE TO RELATED APPLICATION

This application claims the benefit of U.S. Provisional Patent Application No. 63/544,670, filed on Oct. 18, 2023, which is incorporated by reference herein in its entirety.

### STATEMENT REGARDING FEDERALLY SPONSORED RESEARCH

This invention was made with government support under Grant No. EB026710 awarded by the National Institutes of Health. The government has certain rights in the invention.

### BACKGROUND OF THE INVENTION

### 1. Field of the Invention

The present invention generally relates to a network carrying electrical signals. More particularly, the present invention relates to a system and method for minimizing signal loss in connecting a source to a load through an electrical two-port network. The present invention relates to coaxial cables in electrical impedance tomography.

### 2. Description of the Related Art

Electrical Impedance Tomography (EIT) is a promising technology for medical imaging applications that produces images of the body's inner organs from electrical measurements made on its surface. These images represent the distribution of electrical conductivity and permittivity within the body. To perform EIT, multiple electrodes are attached to the body under test and these electrodes are often connected to the system electronics using shielded cables. EIT is noninvasive, radiation-free, has no harmful side effects, and is suitable for patients of any age.

In most EIT systems, one or more known currents are passed through the body and the voltages are measured on the body surface. Thus, the core of the EIT operation is the current source(s) used to pass known currents. To obtain high quality images using EIT, a “high quality” current source is generally needed. The term “high quality” can be defined as the source maintaining a very high output impedance and supplying the desired current to the electrodes throughout the required frequency range. In theory, the output impedance should be infinitely large.

Delivering the desired current to an unknown load and measuring the exact voltage on that load are made more difficult by the conductive cables connecting the electronics to the load (body). The finite current source output impedance, the shunting stray capacitance due to the circuit board and/or wires, and the parallel capacitance and series inductance and resistance of the cable all degrade the performance of the current source. Although using shielded cables adds a significant amount of parasitic capacitance, which most affects the current source high-frequency operation, shields are needed to reduce the interference between the current sources in a multi-source system.

Further, the cable shunt capacitance is most harmful to the high frequency operation of the current source where the impedance presented by the capacitance becomes smaller. Multiple approaches exist for reducing the effect of the cable shunt capacitance, including using an active circuit to drive (guard) the cable shield or using grounded shield with a current source that supplies the additional current that is lost to the capacitance. However, these approaches do not address any series components of the cable.

The use of “active electrodes” is an alternative approach that reduces the effects of cables. Here, the core components of the EIT system, from the current source to the voltage buffer, are placed at the end of the cable with the electrodes that are connected to the patient. This approach reduces the impact of not only the shunt capacitance but the series cable elements by moving the sensitive electronics to the patient side of the cable. Hygiene requirements, however, limit the use of active electrodes, as the electrodes need to be cleaned or sanitized after each patient's use and the inconvenience and cost of having circuit boards directly attached to the patient can make this more difficult. Reusable electronics development has not been practicable, and thus, single-use electrodes are recommended to overcome the hygiene issues. But single-use electrodes increase the system cost dramatically.

Accordingly, improving the performance of a non-active electrode EIT system requires addressing the series components of the coaxial cable. The effect of series elements is most important when measuring low impedance loads at high frequencies. The voltage drop due to the cable series inductance can be significant when compared to the load voltage, making the measured load voltage significantly higher than the actual voltage across the load and decreasing the accuracy of the voltage measurements. It is accordingly to improve the performance of EIT cable signals that the present invention is primarily directed.

### BRIEF SUMMARY OF THE INVENTION

Briefly described, the present invention is for a system and method for minimizing signal loss when connecting a source to a load through a two-port electrical network. The invention modifies the current applied at the input of the two-port network to compensate for losses due to the two-port network and to attain a desired load current without using measurements at the load end of the two-port network. The applied current is adjusted using the measured voltage at the input and knowledge of the two-port parameters found during a calibration process. The invention also provides the voltage at the load side from measurements at the input side of the two-port network. In one embodiment, the two-port network is a coaxial cable.

The invention delivers a desired load current to a load at one end of a coaxial cable from a current source at the other end in coaxial cables carrying signals for electrical impedance tomography (EIT) by addressing both the series and shunt effects of the cables in a system that utilizes an adaptive current source.

The present invention is particularly applicable to systems that can be fully characterized by a set of ABCD two-port parameters. In one embodiment, ABCD parameters can be used to represent the cables in EIT systems. This allows for compensation for the coaxial cable series elements (resistance and inductance) by estimating the load current and calculating the correction factor needed for shunt elements compensation. This allows the minimization of the losses of the coaxial cable.

In one embodiment, the invention models the coaxial cables as a two-port network and minimizes losses when these coaxial cables conducting electrical impedance tomography (EIT) signals by using a control logic. The input to the coaxial cable is driven by an input current source and the output to the coaxial cable delivers an output current to a load. The control logic is configured to adjust the input current source to compensate for losses in the coaxial cable to attain a desired load current by estimating the load current, calculating a correction factor to attain the desired load current, and then modifying the input current source with the correction factor.

The control logic can be further configured to perform a predetermined calibration process. For example, the calculation of the voltage at the load end of the cable from the current applied to and voltage measured at the input of the cable can be calibrated using the measured cable parameters. The control logic can also be further configured to measure a voltage in each input current source and adjust the current for each output current based on the measured voltage. Further, the control logic can be further configured to modify the input current by adjusting the input current using a measured resistance and inductance in each input current source. The control logic further can be configured to compensate for losses from shunt impedance from source to load up to 1 MHz with the shield grounded. Moreover, the control logic can be configured as an electrical circuit or a computer platform.

In an embodiment, the invention includes a method for minimizing losses in multiple coaxial cables conducting electrical impedance tomography (EIT) signals to multiple electrodes by the steps of connecting the input each coaxial cable to an input current source controlled using a control logic with each cable having an output current. The method continues with estimating, at the control logic, the load current for each input current source, calculating, at the control logic, a correction factor to attain a desired load current on each output cable, and then modifying the input current for each output coaxial cable with the correction factor. The method can include the step of performing, at the control logic, a predetermined calibration process.

The present invention therefore provides an advantage in optimizing systems that perform EIT. Further, the present invention is industrially applicable in medical devices that perform EIT for diagnostic and research purposes. Further, the invention provides an advantage in systems that seek to provide a desired current to a load through a cable or other network, measure the voltage on the load, or both. Other advantages and features of the present invention will be apparent to one of skill in the art after review of the present application.

### BRIEF DESCRIPTION OF THE DRAWINGS

FIG. 1 is a schematic diagram of a two-port network.

FIG. 2 is a schematic diagram of cascaded two-port networks with shared voltages and currents.

FIG. 3 is a schematic diagram of one embodiment of the control logic for a two-port network with a load

FIG. 4 is a schematic diagram of one embodiment of a circuit for system for a two-port network of 1 m of RG174 coaxial cable with a 100Ω load.

FIG. 5 is a graph of an adaptive voltage convergence for a 100Ω load.

FIG. 6 is a graph of an adaptive load resistance for a 100Ω load.

FIG. 7 is a graph of an estimation relative error load current and voltage.

FIG. 8 is a graph of experimental results for adaptive voltage convergence for 100Ω load.

FIG. 9A is a graph of an experimental reconstructed measured load resistance.

FIG. 9B is a graph of an experimental reconstructed load resistance relative error.

### DETAILED DESCRIPTION OF THE INVENTION

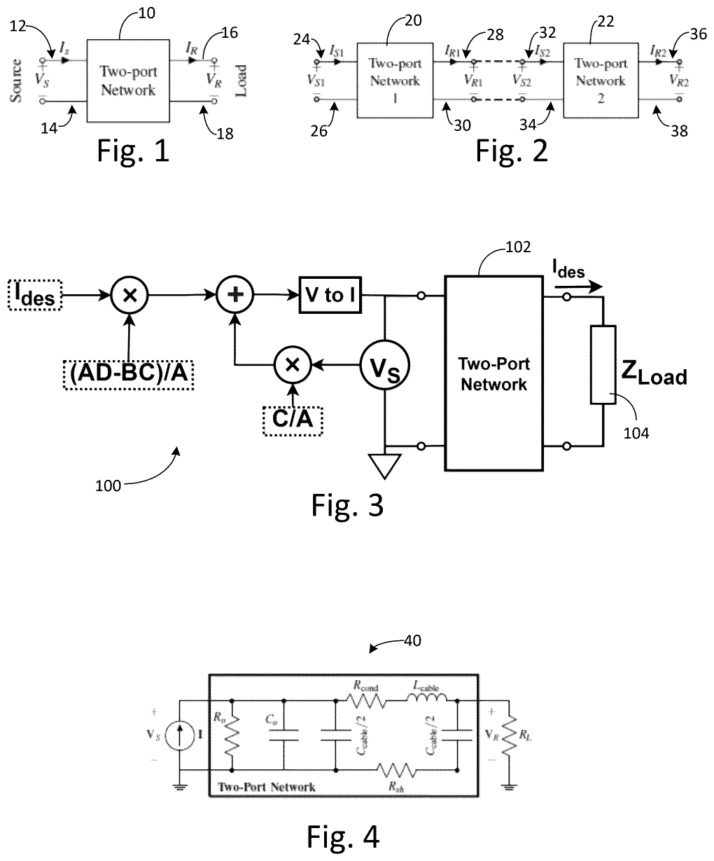

With reference to the figures in which like numerals represent like elements throughout the several views, FIG. 1 is a schematic diagram of a two-port network 10. One embodiment of a two-port network is a coaxial cable. The coaxial cable may be part of an EIT system where it connects system electronics to an electrode. As shown, the source is on the left meaning that the current is supplied from and voltage measurements are made at this end of the cable 12,14. The load is on the right of the figure and it is the goal of the EIT system to apply the desired current to and measure the voltage at this load at 16, 18. As a result of the coaxial cable (two-port network), a relation that transforms the input voltage and current to the output voltage and current is performed making the voltages and currents at the input 12,14 not equal the voltage and currents at the output 16,18.

Thus, the two-port network 10 alters the signals, introducing “losses” in electrical impedance tomography (EIT) signals which can be mitigated by using a control logic, which can be implemented as either hardwired logic, middleware, or a general computer platform. The control logic is configured to compensate for losses introduced by coaxial cable 10 to attain a desired load current (Load) for the output current at 16,18 by estimating the load current, calculating a correction factor to attain the desired load current at the output, and then modifying the input current at 12,14 with the correction factor. Also, the control logic is configured to compute the voltage at 16, 18 from the voltage and current at 12, 14. These steps are further described herein.

The control logic can be further configured to perform a predetermined calibration process to optimize the compensation process. The control logic can also be further configured to measure a voltage at the input to the coaxial cable 12,14 and adjust the input current at 12, 14 to achieved a desired output current at 16, 18 based on the measured voltage at 12, 14. Further, the control logic can be further configured to modify the input current by adjusting the input current using a measured shunt resistance and capacitance in each input current source. The control logic further can be configured to compensate for losses from shunt impedance from source to load up to 1 MHz with the shield grounded.

Herein, several two-port network 10 representations are commonly used, including S-parameters that are used for high frequency circuits, h-parameters that are used for active circuit modeling, and the ABCD parameters that are mainly used for transmission line modeling. The ABCD parameters are well-suited for this application since they relate the voltage and current at the sending (source) side, V_{S} and I_{S} respectively, to the corresponding receiving (load) side voltage and current, V_{R} and I_{R}. The relationship is represented using the ABCD-matrix as:

$$[ \begin{matrix}{V}_{S} \\ {I}_{S}\end{matrix} ] = [ \begin{matrix}A & B \\ C & D\end{matrix} ] [ \begin{matrix}{V}_{R} \\ {I}_{R}\end{matrix} ] . \qquad ( 1 )$$

Once the matrix is known, it is possible to compute the current and voltage at the network output (Load side) from the current and voltage at the network input (Source side) where they can be readily measured using the electronics, enabling our goal of compensating for the cable series and shunt elements.

To design a current source for EIT, the parameter matrix in equation (1) needs to be expanded to include the current source and the coaxial cable. The current source output impedance and the cable can be seen as individual two-port networks each with their own set of ABCD parameters. The two individual two-ports are cascaded with each other as in FIG. 2.

FIG. 2 is a schematic diagram of cascaded two-port networks with shared voltages and currents. There is a first two-port network 20 and a second two-port network 22 in series. The first input current source at 24,26 is in electrical connection with the first two-port network 20 with first output at 28,30. First output 28, 30 is in electrical connection with the second input 32, 34 of the second two-port 22 with second output 36, 38.

The receive side elements for the first two-port network 20 are I_{R1}, V_{R1} are equal to the sending side elements of the following stage I_{S2}, V_{S2}. The overall ABCD matrix is simply the matrix multiplication of each individual ABCD matrix as;

$$[ \begin{matrix}{V}_{S 1} \\ {I}_{S 1}\end{matrix} ] = [ \begin{matrix}{A}_{1} & {B}_{1} \\ {C}_{1} & {D}_{1}\end{matrix} ] [ \begin{matrix}{A}_{2} & {B}_{2} \\ {C}_{2} & {D}_{2}\end{matrix} ] [ \begin{matrix}{V}_{R 2} \\ {I}_{R 2}\end{matrix} ] . \qquad ( 2 )$$

Subscript 1 refers to the first two-port network 20 and subscript 2 refers to two-port network 22.

Since two-port network 20 is the output impedance of a current source with just a shunt impedance, the ABCD parameters matrix is represented by:

$$[ \begin{matrix}1 & 0 \\ Y & 1\end{matrix} ] , \qquad ( 3 )$$

where Y is the output admittance of the current source. So the cascaded matrix in Eq. 2 can be simplified to:

$$[ \begin{matrix}{V}_{S 1} \\ {I}_{S 1}\end{matrix} ] = [ \begin{matrix}{A}_{2} & {B}_{2} \\ {YA}_{2} + {C}_{2} & {YB}_{2} + {D}_{2}\end{matrix} ] [ \begin{matrix}{V}_{R 2} \\ {I}_{R 2}\end{matrix} ] \qquad ( 4 )$$

Eq. 4 provides an entire system characterization. The set of parameters associated with the coaxial cable is represented by two-port network 22 with the ABCD parameters having the subscript 2.

This approach uses the values of the ABCD parameters measured during a calibration process to determine how to adjust the applied current (I_{S} or I_{S1}) to get the desired load current (I_{R} or I_{R2}) and to transform the measured voltage (V_{S} or V_{S1}) to get the voltage at the load (V_{R} or V_{R2}). The ABCD matrix represents both the series and shunt impedances of the coaxial cable and, also, the current source output impedance which can be represented as an output resistance, Rₒ in parallel with an output capacitance, Cₒ.

Solving Eq. 1 to eliminate I_{R} produces Eq. 5 which gives the load voltage V_{R} in terms of quantities measurable at the source, I_{S} and V_{S′}:

$${V}_{R} = \frac{{DV}_{S} - {BI}_{S}}{AD - BC} . \qquad ( 5 )$$

Likewise, solving Eq. 1 to eliminate V_{R} produces Eq. 6 which gives the load current in terms of I_{S} and V_{S}:

$${I}_{R} = \frac{{AI}_{S} - {CV}_{S}}{AD - BC} . \qquad ( 6 )$$

While it is sufficient to be able to find the actual load voltage, V_{R}, after the current is applied, the goal is to use the ABCD parameters to apply the appropriate current to produce a desired load current, I_{des}, at the load, i.e. I_{R}=I_{des}. The current source adjusts the output current to account for current lost in the shunt output and stray impedance by multiplying the desired current by a scale factor S_{scale}. The same approach is taken here, meaning that the current on the source side is set to:

$${I}_{S} = {S}_{scale} {I}_{des} . \qquad ( 7 )$$

Substituting Eq. 7 and I_{R}=I_{des} into Eq. 6 and solving for S_{scale} gives:

$${S}_{scale} = \frac{AD - BC}{A} + \frac{{CV}_{S}}{{AI}_{des}} . \qquad ( 8 )$$

This equation is used to adjust the applied current based on the voltage observed at the source side of the cable and the current that is desired at the load end of the cable. Since V_{S} is affected by the applied current, forcing I_{R}=I_{des} is an iterative process.

FIG. 3 is a schematic diagram 100 of one embodiment of the control logic that applies the needed current to the two port network 102 that results in I_{des} flowing in Z_{Load} 104 at the output of 102. The input to the control logic is the value of the desired load current I_{des}. The block labeled “V to I” converts voltage to a proportional current and the block with V_{S} in a circle is a voltmeter that measures the voltage on the left side of the two-port network. The control logic implements Eq. 8 and the current applied to the left side of the two-port 102 where:

$${I}_{S} = {S}_{scale} {I}_{des} = [ \frac{AD - BC}{A} ] {I}_{des} + \frac{C}{A} {V}_{S}$$

Simulations were performed using a combination of LTspice® and MATLAB and experimental results were obtained to validate the proposed approach. The model for 1 m of RG174 coaxial cable with nominal values was used to connect the adaptive current source to a 100Ω load as in FIG. 4. FIG. 4 is a schematic diagram 40 of one embodiment of a cascaded current source output impedance and cable model.

A current source output impedance model with output resistance of Rₒ=100 kΩ in parallel with output capacitance of Cₒ=40 pF was included in the model. The parameters for the RG174 cable and the source output impedance are shown in Table 1 below. The calibration process for determining the source output impedance is described in detail in A. Abdelwahab, O. Rajabi Shishvan, and G. J. Saulnier. Performance of an adaptive current source for EIT driving loads through a shielded coaxial cable. In 2020 42nd Annual International Conference of the IEEE Engineering in Medicine Biology Society (EMBC), pages 1448-1451, 2020, the entirety of which is hereby incorporated herein by this reference.

The LTspice simulations were run over a frequency range of 50 kHz-1 MHz. Since the simulation environment allows us to measure the current and voltage at both sides of the cable, the ABCD parameters were measured at each frequency by replacing the load with an open-circuit to measure the voltage ratio A=V_{S}/V_{R} and admittance C=I_{S}/V_{R}. Similarly, the load was replaced with a short-circuit to measure the impedance B=V_{S}/I_{R} and current ratio D=I_{S}/I_{R}. The resulting values were transferred to MATLAB for processing.

TABLE 1
Simulation parameters

| Parameter | Value |
|---|---|
| R [symbol] | 0.3 Ω/m |
| C [symbol] | 101 pH/m |
| I [symbol] | 0.25 μH/m |
| R [symbol] | 0.035 Ω/m |
| Characteristic Impedance | 50 Ω |
| Source output Impedance | 100 kΩ/40 pF |

[symbol]

indicates data missing or illegible when filed

Once the parameters were calculated, a 100Ω load was connected for the actual measurement process. The input current source was set to apply the desired current I_{des} and the voltage V_{S} measured. Then using the pre-calculated ABCD parameter matrix along with the known desired current I_{des} and measured the voltage V_{S}, the correction scale S_{scale} was calculated at each frequency using Eq. 8. The current source output current was then multiplied by S_{scale} to produce the new applied current and the process was repeated until source side voltage V_{S} was fully converged.

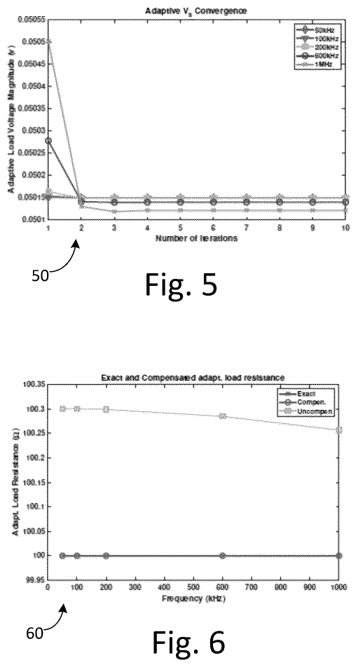

FIG. 5 is a graph 50 of an adaptive voltage convergence for a 100Ω load. FIG. 5 shows the voltage magnitude as a function of iteration for several frequencies, indicating that the result converges within a few iterations. As expected, the final values differ more from the initial values as frequency increases due to the fact that a larger adjustment in applied current is needed to compensate for the lower impedance presented by the shunt capacitance.

The converged value of V_{S} was used in Eq. 5 to determine the load voltage V_{R}. The exact values for load voltage and current are measured directly and represent the ground truth for comparison with the estimated values. FIG. 6 is a graph 60 of an adaptive load resistance for a 100Ω load. FIG. 6 shows the reconstructed values of the load resistance for three configurations. “Exact” uses the voltage and current measured at the load to compute the value of the 100Ω load resistance. “Compensated” utilizes the estimation of V_{R} obtained using V_{S} and the desired current I_{des} to compute the load resistance. The “Exact” and “Compensated” curves nearly coincide. Finally, the load resistance for “Uncompensated” is calculated by measuring the voltage and current at the source side V_{S} and I_{S}. The results show 0.25-0.3Ω error in estimating the 100Ω resistance in the uncompensated case, but a nearly perfect matching with the exact for the compensated case.

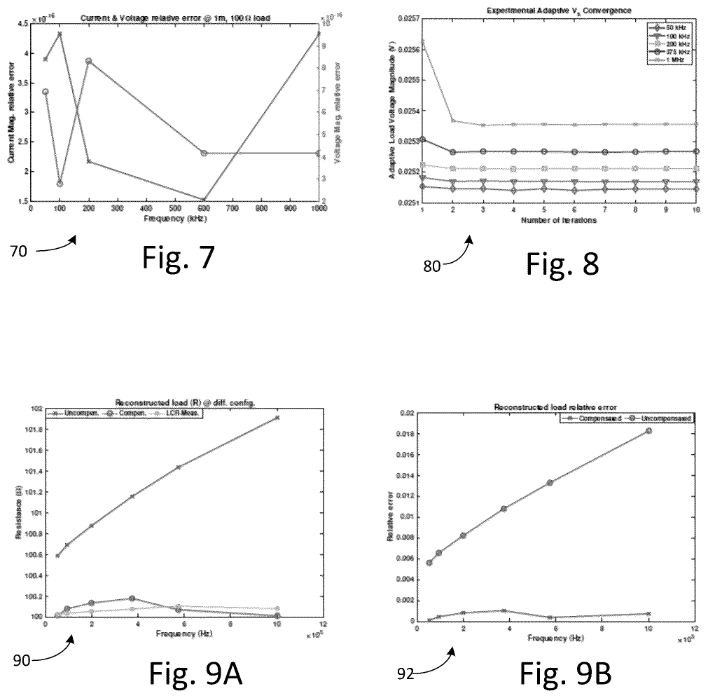

The relative errors between the ground truth and estimated values of currents and voltage in the compensated case are shown in FIG. 7. FIG. 7 is a graph 70 of an estimation relative error load current and voltage. The curve with “x” markers is the relative error in the current and the curve with “o” markers is the relative error in the voltage. The errors are in the range of 10⁻¹⁶ and are due to the truncation of the numerical values taken from the LTspice® simulation. As expected, the algorithm provides nearly perfect compensation for the cable effects when provided with nearly perfect ABCD parameters and voltage readings.

For initial verification of the compensation approach, one can set the load to be purely resistive and use a relatively small load resistance since it results in greater sensitivity to the series elements of the cable. However, the EIT systems often encounter biological loads that are complex, with both resistive and capacitive elements, so it is important to note that this compensation approach is valid for all load values, but the effect is more obvious with a low-value load.

For additional verification of the approach, experiments were performed using the same conditions as in the simulation. A 100Ω precision resistor (Vishay Foil Resistors VFCP Series, ±0.02%, ±0.2 ppm/° C.) load and 1 m of RG174 coaxial cable were used. The current generation and voltage measurements were done using the ACT5 EIT system described in O. R. Shishvan, A. Abdelwahab, N. B. da Rosa, G. J. Saulnier, J. L. Mueller, J. Newell, D. Isaacson. ACT5 Electrical Impedance Tomography System. IEEE Trans Biomed Eng. 2024 January; 71 (1): 227-236. doi: 10.1109/TBME.2023.3295771. The SOURCETRONIC ST2829C precision LCR meter was used for measuring the cable series/shunt components as well as the 100Ω load resistor to be used as ground truth for evaluating the compensation approach. The LCR meter has a test frequency range of [20 Hz-1 MHz] with a basic accuracy of 0.05%.

After fully characterizing the current source and the cable, the cascaded ABCD matrix is calculated for the excitation frequency range [50 kHz-1 MHz]. The same sequence followed in the simulation was performed for the experiments. A desired load current I_{des} of 0.25 mA was applied, the voltage V_{S} measured and, using the ABCD parameters matrix, the current correction S_{scale} was calculated at each excitation frequency using Eq. 8. The current source output was then scaled by S_{scale} to produce the corrected applied current which compensates for all shunt components. This process was repeated until source side voltage V_{S} had fully converged as in FIG. 8.

FIG. 8 is a graph 80 of experimental results for adaptive voltage convergence for 100Ω load. The convergence results agree well with the simulation results, requiring only a few iterations to reach a steady-state. The converged value of V_{S} was used in Eqs. 5 and 6 to determine the load voltage and current, V_{R} and I_{R} respectively, and hence reconstruct the load value. The LCR-measured load values are considered the ground truth for evaluating the proposed approach.

The reconstructed load resistance values using both the compensated/uncompensated voltages are shown in FIG. 9A. FIG. 9A is a graph 90 of an experimental reconstructed measured load resistance. The plot in FIG. 9A shows good agreement between the load values reconstructed using the compensated voltage and the LCR-measured values.

FIG. 9B is a graph 92 of an experimental reconstructed load resistance relative error. FIG. 9B shows that the relative error with compensation is 0.1% or lower across the frequency range while the peak error without compensation is 1.825%. The near zero error obtained through simulation is not expected in the practical implementation due to limitations in the measurement accuracy for the impedance values used to construct the ABCD matrices and any unmodeled series resistance or inductance from the board traces and the connectors.

The corresponding structures, materials, acts, and equivalents of all means or step plus function elements in the claims below, if any, are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of one or more aspects of the invention and the practical application, and to enable others of ordinary skill in the art to understand one or more aspects of the invention for various embodiments with various modifications as are suited to the particular use contemplated.

What is claimed is:

1. A system for minimizing the effect of losses in coaxial cables conducting electrical impedance tomography (EIT) signals, comprising:
a control logic;
an input to the coaxial cable in electrical connection with the control logic, with the input having an input current source; and
an output to the coaxial cable having an output current,
wherein the control logic configured to compensate for losses in the coaxial cable to attain a desired load current for the output current the coaxial cable by:
estimating the load current;
calculating a correction factor to attain the desired load current on each output cable; and
modifying the input current for each input coaxial cable with the correction factor.

2. The system of claim 1, wherein the control logic is further configured to perform a predetermined calibration process to compute the voltage on the load from the voltage at the input current.

3. The system of claim 2, wherein the control logic further configured to;
measure a voltage in each input current source; and
adjust the input current source based on the measured voltage.

4. The system of claim 3, wherein the control logic further configured to modify the output current by adjusting the input current source.

5. The system of claim 1, wherein the control logic further configured to compensate for losses from shunt impedance from an input current source up to 1 MHz.

6. The system of claim 1, wherein the control logic is an electrical circuit.

7. The system of claim 1, wherein the control logic is a computer platform.

8. A method for minimizing losses in coaxial cables conducting electrical impedance tomography (EIT) signals, comprising:
connecting at least two input coaxial cables to a control logic;
connecting at least two output coaxial cables the control logic, each cable having an output current,
providing an input current source for each of input coaxial cables to the control logic;
estimating, at the control logic, a load current for each output current source;
calculating, at the control logic, a correction factor to attain a desired load current on each output cable; and
modifying the input current for each input coaxial cable with the correction factor.

9. The method of claim 8, performing, at the control logic, a predetermined calibration process to compute the voltage on each load from the voltage at each input current.

10. The method of claim 9, further including:
measuring a voltage in each input current source; and
adjusting the current for each input current based on the measured voltage.

11. The method of claim 8, wherein modifying the input current is adjusting the input current using a measured resistance and inductance in each input current source.

12. The method of claim 8, further including compensating for losses from shunt impedance from an input current source up to 1 MHz.

13. The method of claim 8, further including:
connecting at least two second input coaxial cables to a second control logic;
connecting at least two second output coaxial cables the second control logic, each second output coaxial cable having a second output current,
providing a second input current source for each of the second input coaxial cables to the second control logic;
estimating, at the second control logic, the load current for each second output current source;
calculating, at the second control logic, a second correction factor to attain a desired load current on each second output coaxial cable; and
modifying the input current for each second output coaxial cable with the second correction factor.

14. The method of claim 8, wherein the input coaxial cables and output coaxial cables are connected to an electrical circuit.

15. The method of claim 8, wherein the input coaxial cables and output coaxial cables are connected to a computer platform.

16. A system for applying a desired current to a load connected to a source by an electrical two-port network comprising:
control logic;
an input to the two-port network in electrical connection with the control logic, with the input having a current source; and
an output having an output current into the load,
wherein the control logic is configured to compensate for the losses in the two-port network to attain a desired load current by:
estimating the load current using measurements made at the input to the two-port network;
calculating a correction factor to attain the desired load current; and
modifying the input current with the correction factor.

17. The system of claim 16, wherein the control logic is further configured to perform a predetermined calibration process to compute the voltage on the load from the voltage at the input current.

18. The system of claim 16, where the two-port network is a coaxial cable.

19. The system of claim 16 where multiple current sources are connected to multiple loads through multiple two-port networks and multiple instances of the control logic are used to deliver the desired currents to each load.
